(19) United States
(12) Patent Application Publication
(10) Pub. No.: US 2010/0056750 A1
(43) Pub. Date: Mar. 4, 2010

(54) PROCESS FOR THE SYNTHESIS OF HALOGENATED AROMATIC DIACIDS

(76) Inventor: Joachim C. Ritter, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/516,011

(22) PCT Filed: Dec. 18, 2007
(86) PCT No.: PCT/US07/25798

§ 371 Date: May 22, 2009

Related U.S. Application Data
(60) Provisional application No. 60/876,576, filed on Dec. 21, 2006.

(51) Int. Cl. C07C 51/16; C08G 63/00

(52) U.S. Cl. 528/308.8; 562/409

(57) ABSTRACT

The production of high-purity halogenated aromatic diacids from halogenated dimethylbenzene by oxidation with an oxygen-containing gas is conducted using a four-component catalyst system and a two-stage temperature process.

This application claims the benefit of U.S. Provisional Application No. 60/876,576, filed 21 Dec. 2006, which is incorporated in its entirety as a part hereof for all purposes.

### TECHNICAL FIELD

This invention relates to the manufacture of halogenated aromatic diacids, which are used industrially as compounds and as components in the synthesis of a variety of useful materials.

### BACKGROUND

Halogenated aromatic diacids, as described generally by the structure of Formula (I)

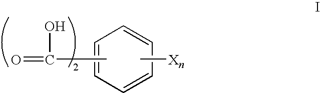

where X=Cl, Br or I, and n=1 or 2, have a variety of industrial uses, for example, as flame retardants and as intermediates in the production of pigments, dyestuffs, herbicides and polymers.

One such compound, 2,5-dibromoterephthalic acid, as described generally by the structure of Formula (II),

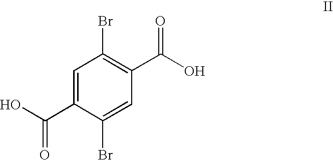

has been produced by the oxidation of 2,5-dibromo-1,4-dimethylbenzene in acetic acid using a Co/Mn II catalyst, as described in GB 1,238,224. Yields are up to about 70% in a batch process and 85% in a semicontinuous operation mode. U.S. Pat. No. 3,894,079 describes the bromination of terephthalic acid in chlorosulfonic or fluorosulfonic acid with sulfur trioxide in the presence of iodine and bromine, with demonstrated yields of about 44-70% 2,5-dibromoteretphthalic acid. Other known methods use KMnO₄ HNO₃ or Na₂CrO₇ as an oxidant in stoichiometric quantities.

As described in Canadian Patent 1,173,458, 2,5-dichloroterephthalic acid has been produced by the oxidation of 2,5-dichloro-1,4-dimethylbenzene in an acetic acid solvent with an oxygen-containing gas in the presence of a catalyst system at a reaction temperature of from about 150° C. to about 300° C. and a reaction pressure of from about 100 to about 1000 psig, wherein the catalyst systems contains a cobalt compound catalyst in an amount of from about 0.02 to about 2 weight percent, a manganese compound co-catalyst in an amount of from about 0.02 to about 2 weight percent, and a bromine compound promoter, in an amount of from about 0.03 to about 8 weight percent, said weight percents being based on the weight of the solvent.

As described in U.S. Pat. Nos. 3,142,701, 2,5-diiodoterephthalic acid, 2,5-dichloroterephthalic acid, and 2,5-dibromoterephthalic acid have been produced by gradually introducing the desired halogen (bromine, chlorine, or iodine) into an oleum (i.e. SO₃/HSO₄) solution of terephthalic acid, then raising the temperature to about 50-75° C. and heating for several hours.

Despite processes for the production of a halogenated aromatic diacid as described above, a need remains for such a process that has a desirably high selectivity, yield, product purity, and ease of recovery.

### SUMMARY

The inventions disclosed herein include processes for the preparation of halogenated aromatic diacids, processes for the preparation of products into which halogenated aromatic diacids can be converted, the use of such processes, and the products obtained and obtainable by such processes.

One embodiment of the processes hereof provides a process for the preparation of a halogenated aromatic diacid, as described by the structure of Formula (I)

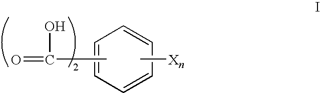

wherein X is Cl, Br or I, and n=1 or 2, by

(a) providing a solution of a catalyst system in a solvent, the catalyst system including a cobalt compound, a manganese compound, a zirconium compound, and a bromine compound;

(b) contacting the solution with a halogenated dimethylbenzene, as described by the structure of Formula (III)

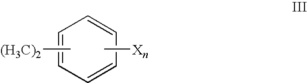

to form a reaction mixture;

(c) stirring the reaction mixture while injecting an oxygen-containing gas therein;

(d) heating the reaction mixture at a first temperature to oxidize one of the two methyl groups of the halogenated dimethylbenzene to produce a compound as described by the structure of Formula (IV); and

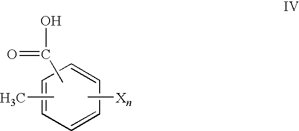

(e) heating the reaction mixture at a second temperature that is higher than the first temperature to oxidize the methyl group in the compound of Formula (IV) to produce a halogenated aromatic diacid.

Another embodiment of the processes hereof involves a process for preparing a halogenated aromatic diacid that further includes a step of subjecting the halogenated aromatic diacid to a reaction (including a multi-step reaction) to prepare therefrom a compound, monomer, oligomer or polymer.

### DETAILED DESCRIPTION

The production of halogenated aromatic diacids, as described generally by the structure of Formula (I), from halogenated dimethylbenzenes by oxidation with an oxygen-containing gas is conducted using a four-component catalyst system and a two-stage temperature process. In several specific embodiments, the halogenated aromatic diacid obtained from a process hereof may be 2,5-dibromoterephthalic acid, 2,5-dichloroterephthalic acid, 2-bromoterephthalic acid, 2-chloroterephthalic acid, 2,4-dibromoisophthalic acid, 2,4-dichloroisophthalic acid, 2-bromoisophthalic acid, 2-chloroisophthalic acid, 4-bromoisophthalic acid, 4-chloroisophthalic acid, 5-bromoisophthalic acid, or 5-chloroisophthalic acid.

The catalyst system used herein may contain a cobalt compound, a manganese compound, a zirconium compound, and a bromine compound. Preferably, the mole ratio of cobalt compound:manganese compound:zirconium compound:bromine compound is about 1:1-1.5:0.05-0.2:1-3.

Cobalt compounds suitable for use in the catalyst system hereof include cobalt salts such as cobalt acetate, cobalt naphthenate, cobalt 2-ethylhexanoate, cobalt bromide and mixtures thereof. A cobalt compound is preferably used in an amount of from about 0.1 to about 5 mol % based on moles of halogenated dimethylbenzene. A preferred cobalt compound is cobalt acetate.

Manganese compounds suitable for use in the catalyst system hereof include manganese salts such as manganese acetate, manganese naphthenate, manganese 2-ethylhexanoate, manganese bromide and mixtures thereof. A manganese compound is preferably used in an amount of from about 0.1 to about 5 mol % based on moles of halogenated dimethylbenzene. A preferred manganese compound is manganese acetate.

Zirconium compounds suitable for use in the catalyst system hereof include zirconium (IV) salts such as zirconium acetate, zirconium naphthenate, zirconium 2-ethylhexanoate, zirconium bromide and mixtures thereof. A zirconium compound is preferably used in an amount of from about 0.01 to about 0.5 mol % based on moles of halogenated dimethylbenzene. A preferred zirconium compound catalyst is zirconium acetate.

Bromine compounds suitable for use in the catalyst system hereof include brominated salts such as sodium bromide, potassium bromide, hydrogen bromide, bromine, cobalt bromide, manganese bromide, zirconium bromide, tetrabromoethane and mixtures thereof. A bromine compound is preferably used in an amount from about 0.2 to about 8 mol % based on moles of halogenated dimethylbenzene. A preferred bromine compound is sodium or potassium bromide. Without limiting the invention to any particular theory of operation, it is believed that the bromine compound functions as a promoter within the catalyst system.

A preferred catalyst system contains cobalt acetate, manganese acetate, zirconium acetate, and sodium bromide in a molar ratio of cobalt acetate:manganese acetate:zirconium acetate:sodium bromide of about 1:1-1.5:0.05-0.2: 1-3, and more preferably about 1:1:0.1:2.

Various cobalt, manganese, zirconium and bromine compounds suitable for use in the catalyst system hereof are available commercially from suppliers such as Alfa Aesar (Ward Hill, Mass.), City Chemical (West Haven, Conn.), Fisher Scientific (Fairlawn, N.J.), Sigma-Aldrich (St. Louis, Mo.) or Stanford Materials (Aliso Viejo, Calif.).

A solution of the catalyst system is prepared in a solvent such as a monocarboxylic acid solvent. Examples of monocarboxylic acids suitable for use as a solvent for such purpose include without limitation aliphatic monocarboxylic acids having 2 to 8 carbon atoms (for example, acetic acid, propionic acid, butyric acid, and the like), benzoic acid, bromobenzoic acids, and phenylacetic acid. Aliphatic monocarboxylic acids having 2 to 8 carbon atoms are preferred, and acetic acid is more preferred.

A solution of the catalyst system is contacted with a halogenated dimethylbenzene. The amount of solvent used is not critical and can vary over a wide range. Typically, the relative amounts of solvent and halogenated dimethylbenzene will be in the range of from about 15 to about 50 grams of halogenated dimethylbenzene per hundred grams of solvent, such as a monocarboxylic acid solvent.

A process hereof may be run as a two-stage liquid phase oxidation reaction wherein a catalyst system containing cobalt, manganese, zirconium and bromine compounds is used to catalyze the oxidation of the alkyl substituents on the halogenated dimethylbenzene to carboxylic acid substituents. An oxygen-containing gas, such as a gas containing molecular oxygen, supplies the oxygen for the oxidation reaction. The process may be conducted, for example, in an enclosed reactor with pressure maintained between about 100 psi (0.7 MPa) and about 1500 psi (10.3 MPa), preferably between 300 psi (2.1 MPa) and about 500 psi (3.4 MPa). The process may be conducted as a batch process, a semi-continuous process, or a continuous process using techniques known in the art for conducting liquid phase oxidations.

In a batch process, the halogenated dimethylbenzene is combined with a solution of catalyst system in the reaction vessel at a temperature ranging from ambient to a first reaction temperature, and the oxygen-containing gas is injected into the closed reaction vessel. The injection of the oxygen-containing gas may supply all or part of the desired mixing, or other means to provide mixing may be used instead or in addition as efficient mixing allows for a sufficient supply of dissolved oxygen in the reaction solution.

The reaction mixture is then heated at a first reaction temperature, which may be between about 120° C. and about 150° C., while the reaction mixture is continuously stirred and an oxygen-containing gas is continuously injected, to oxidize the more reactive first methyl group to a carboxylic acid group, —COOH [see the structure of Formula (IV)]. The oxygen-containing gas employed can vary from pure oxygen to a gas containing about 0.1 percent by weight molecular oxygen, with the remaining gas being a ballast gas, such as nitrogen, that is inert in the liquid phase oxidation. For reasons of economy, the source of molecular oxygen is frequently air. The specific time during which this phase of the oxidation is conducted will depend on the temperature of the solution, the amount of catalyst, the pressure and the extent of mixing. Typically, from about 0.5 to about 5 hours is consumed during this step. The oxygen-containing gas may be introduced by any convenient, known means such as a gas-dispersing stirrer or a valved inlet for compressed gas injection.

In the next stage of the process, the reaction mixture is heated at a second temperature that is higher than the first temperature while the solution is continuously stirred and oxygen-containing gas is continuously injected therein. The second temperature may be between about 150° C. and about 180° C. This will oxidize the remaining methyl group to a carboxylic acid group, —COOH, to produce the desired halogenated aromatic diacid [as described generally by the structure of Formula (I)]. The oxygen-containing gas may contain about 15 wt % to 100 wt % oxygen, but is, again for convenience, typically air. The second reaction temperature may be about 20 to about 30° C. higher than the first reaction temperature. The specific time during which this phase of the oxidation is conducted will depend on the temperature of the solution, the amount of catalyst, the pressure and the extent of mixing. Typically, from about 1 to about 15 hours is consumed during this step.

The process hereof may be conducted in a continuous manner wherein the reaction components comprising the halogenated dimethylbenzene feedstock, catalyst system, source of molecular oxygen, and solvent are continuously added to selected sites in a first oxidation reaction zone under predetermined reaction conditions and addition rates, including a first oxidation temperature. In a continuous oxidation process, a reaction product mixture containing the partially oxidized halogenated dimethylbenzene [Formula (IV)] may be continuously removed from the first oxidation reaction It zone and fed to a second reaction zone at a second oxidation reaction temperature. The reaction product mixture containing the desired halogenated aromatic diacid [Formula (I)] is then typically continuously removed from the second reaction zone.

The reaction mixture is then cooled or allowed to cool, and the precipitated product is recovered by any convenient means known in the art, typically simple suction filtration.

The catalyst system hereof, the staged temperature approach, and the addition of molecular oxygen throughout the process together appear to result in higher selectivity, yield, and purity of the product. Byproduct formation appears to be minimized as a consequence of avoiding low oxygen concentrations throughout the process. Consequently, product yield and purity are improved.

As used herein, the term “selectivity” for a product P denotes the molar fraction or molar percentage of P in the final product mix. As used herein, the term “conversion” denotes to how much reactant was used up as a fraction or percentage of the theoretical amount. The conversion times the selectivity thus equals the maximum “yield” of P; the actual yield, also referred to as “net yield,” will normally be somewhat less than this because of sample losses incurred in the course of activities such as isolating, handling, drying, and the like. As used herein, the term “purity” denotes what percentage of the in-hand, isolated sample is actually the specified substance.

The halogenated aromatic diacid product hereof may, as desired, be isolated and recovered as described above. It may also be subjected with or without recovery from the reaction mixture to further steps to convert it to another product such as another compound (e.g. a monomer), or ultimately an oligomer or a polymer. Another embodiment of a process hereof thus provides a process for converting a halogenated aromatic diacid, through a reaction (including a multi-step reaction), into another compound, or into an oligomer or a polymer. A halogenated aromatic diacid may be made by a process such as described above, and then converted, for example, into a compound such as a dihydroxyterephthalic acid or a dialkoxyterephthalic acid. A halogenated aromatic diacid may be converted into a dihydroxyterephthalic acid or a dialkoxyterephthalic acid by the processes disclosed in U.S. application Ser. No. 11/604,935, which is incorporated in its entirety as a part hereof for all purposes.

In a multi-step process, the dihydroxyterephthalic acid or a dialkoxyterephthalic acid so produced may in turn be subjected to a polymerization reaction to prepare an oligomer or polymer therefrom, such as those having one or more of ester functionality, ether functionality, amide functionality, imide functionality, imidazole functionality, carbonate functionality, acrylate functionality, epoxide functionality, urethane functionality, acetal functionality, or anhydride functionality, or a pyridobisimidazole-2,6-diyl(2,5-dihydroxy-p-phenylene) polymer.

A dihydroxyterephthalic acid or a dialkoxyterephthalic acid (and thus ultimately a halogenated aromatic diacid as its precursor) may, for example, be converted into a polyester by reaction with either diethylene glycol or triethylene glycol in the presence of 0.1%; of ZN₃(BO₃)₂ in 1-methylnaphthalene under nitrogen, as disclosed in U.S. Pat. No. 3,047,536 (which is incorporated in its entirety as a part hereof for all purposes). Similarly, a 2,5-dihydroxyterephthalic acid is disclosed as suitable for copolymerization with a dibasic acid and a glycol to prepare a heat-stabilized polyester in U.S. Pat. No. 3,227,680 (which is incorporated in its entirety as a part hereof for all purposes), wherein representative conditions involve forming a prepolymer in the presence of titanium tetraisopropoxide in butanol at 200˜250° C., followed by solid-phase polymerization at 280° C. at a pressure of 0.08 mm Hg.

A 2,5-dihydroxyterephthalic acid (and thus ultimately a halogenated aromatic diacid as its precursor) may also be converted into a polymer by reaction with the trihydrochloride-monohydrate of tetraminopyridine in strong polyphosphoric acid under slow heating above 100° C. up to about 180° C. under reduced pressure, followed by precipitation in water, as disclosed in U.S. Pat. No. 5,674,969 (which is incorporated in its entirety as a part hereof for all purposes); or by mixing the monomers at a temperature from about 50° C. to about 110° C., and then 145° C. to form an oligomer, and then-reacting the oligomer at a temperature of about 160° C. to about 250° C. as disclosed in U.S. Provisional Application No. 60/665,737, filed Mar. 28, 2005 (which is incorporated in its entirety as a part hereof for all purposes), published as WO 2006/104974. The polymer that may be so produced may be a pyridobisimidazole-2,6-diyl(2,5-dihydroxy-p-phenylene) polymer such as a poly(1,4-(2,5-dihydroxy) phenylene-2,6-pyrido[2,3-d: 5,6-d′]bisimidazole) polymer. The pyridobisimidazole portion thereof may, however, be replaced by any or more of a benzobisimidazole, benzobisthiazole, benzobisoxazole, pyridobisthiazole and a pyridobisoxazole; and the 2,5-dihydroxy-p-phenylene portion thereof may be replaced by the derivative of one or more of isophthalic acid, terephthalic acid, 2,5-pyridine dicarboxylic acid, 2,6-naphthalene dicarboxylic acid, 4,4′-diphenyl dicarboxylic acid, 2,6-quinoline dicarboxylic acid, and 2,6-bis(4-carboxyphenyl)pyridobisimidazole.

### EXAMPLES

The advantageous attributes and effects of the processes hereof may be seen in a series of examples (Examples 1˜8), as described below. The embodiments of these processes on which the examples are based are illustrative only, and the selection of those embodiments to illustrate the invention does not indicate that conditions, arrangements, approaches, techniques, configurations or reactants not described in these examples are not suitable for practicing these processes, or that subject matter not described in these examples is excluded from the scope of the appended claims and equivalents thereof.

The following materials were used in the examples. All commercial reagents were used as received.

2,5-dibromo-1,4-dimethylbenzene (98% purity), 2-bromo-1,4-dimethylbenzene (99% purity), 2-chloro-1,4-dimethylbenzene (99% purity), 2,5-dichloro-1,4-dimethylbenzene (97% purity), Co(OAc)₂.4H₂O, Mn(OAc)₂.4H₂O, Zr(OAc)₄, and NaBr were obtained from the Aldrich Chemical Company (Milwaukee, Wis., USA).

The meaning of abbreviations is as follows: “DBTA” means 2,5-dibromoterephthalic acid, “DBX” means 2,5-dibromo-1,4-dimethylbenzene, “OAc” means acetate (CH₃COO⁻), “h” means hour(s), “g” means gram(s), “mmol” means millimole(s), “MPa” means megapascal(s), “wt %” means weight percent (age), “psig” means pounds per square inch gage, and “NMR” means nuclear magnetic resonance spectroscopy.

### Example 1

This example illustrates the production of 2,5-dibromoterephthalic acid from 2,5-dibromo-1,4-dimethylbenzene.

In a stirred autoclave with internal cooling coil and reflux condenser, 2,5-dibromo-1,4-dimethylbenzene (372 mmol) was combined with a solution containing Co(OAc)₂.4H₂O (2.5 mmol), Mn(OAc)₂.4H₂O (2.5 mmol), Zr(OAc)₄ (0.25 mmol), and NaBr (5 mmol) in 500 g of 97% acetic acid. The mixture was stirred at a constant rate using a gas dispersing stirrer for better gas mixing and the mixture was heated to 150° C. for 2 h (this stage is noted as “T-1” in Table 1), followed by increasing the temperature to 180° C. for 4 h (this stage is noted as “T-2” in Table 1). While the reaction was heating, air was continuously blown through the system with 400 psig (2.76 MPa) back pressure. After reaction completion, the pressure was released and the reactor was allowed to cool to 50° C. The product was discharged, rinsing the reactor twice with 50 g acetic acid to collect further product. The white solid was collected via suction filtration, washed with water, and dried under vacuum to yield 310 g (84%) of the product 2,5-dibromoterephthalic acid as a white solid with a purity of 99%, as determined by ¹H NMR.

### Examples 2-5

These examples illustrate the effect of varying the stages, times and temperatures on 2,5-dibromoterephthalic acid net yield and purity. Examples 2-5 were carried out using the procedure of Example 1 except as noted in Table 1. The product 2,5-dibromoterephthalic acid in each case was a white solid with a purity of at least 99 mol %.

TABLE 1

| | | Acetate Catalyst | | | | | | Product | |
|---|---|---|---|---|---|---|---|---|---|
| | DBX | (mmol) | | | NaBr | T-1 | T-2 | Purity | Net Yield |
| Example | (mmol) | Co | Mn | Zr | (mmol) | (° C., time) | (° C., time) | (mol % DBTA) | (mol %) |
| 1 | 371 | 2.5 | 2.5 | 0.25 | 5 | 150, 2 h | 180, 4 h | 99% | 81% |
| 2 | 371 | 2.5 | 2.5 | 0.25 | 5 | 120, 2 h | 180, 9 h | 99% | 78% |
| 3 | 371 | 2.5 | 2.5 | 0.25 | 5 | 150, 2 h | 180, 10 h | 99% | 92% |
| 4 | 743 | 5 | 5 | 0.5 | 10 | 150, 2 h | 180, 8 h | 99% | 76% |
| 5 | 743 | 5 | 5 | 0.5 | 10 | 150, 2 h | 180, 4 h | 95% | 86% |

### Example 6

This example illustrates the production of 2-bromoterephthalic acid from 2-bromo-1,4-dimethylbenzene.

In a stirred autoclave with internal cooling coil and reflux condenser, 2-bromo-1,4-dimethylbenzene (541 mmol) was combined with a solution containing Co(OAc)₂.4H₂O (0.625 mmol), Mn(OAc)₂.4H₂O (0.625 mmol), Zr(OAc)₄ (0.15 mmol), and NaBr (0.525 mmol) in 500 g of 97% acetic acid. The mixture was stirred at a constant rate using a gas dispersing stirrer for better gas mixing and the mixture was heated to 150° C. for 2 h followed by increasing the temperature to 180° C. for 4 h. While the reaction was heating, air was continuously blown through the system with 400 psig (2.76 MPa) back pressure. After reaction completion, the pressure was released and the reactor was allowed to cool to 50° C. The product was discharged, rinsing the reactor twice with 50 g acetic acid to collect further product. The white solid was collected via suction filtration, washed with water, and dried under vacuum to yield 113 g (85%) of the product 2-bromoterephthalic acid as a white solid with a purity of 99%, as determined by ¹H NMR.

### Example 7

This example illustrates the production of 2-chloroterephthalic acid from 2-chloro-1,4-dimethylbenzene. The example was carried out as in Example 6 except that 2-chloro-1,4-dimethylbenzene was used in place of 2-bromo-1,4-dimethylbenzene. Filtration and drying under vacuum yielded 45 g (42%) of the product 2-chloroterephthalic acid as a white solid with a purity >99% as determined by ¹H NMR.

### Example 8

This example illustrates the production of 2,5-dichloroterephthalic acid from 2,5-dichloro-1,4-dimethylbenzene. The example was carried out as in Example 6 except that 2,5-dichloro-1,4-dimethylbenzene (571 mmol) was used in place of 2-bromo-1,4-dimethylbenzene. Filtration and drying under vacuum yielded 115 g (86%) of the product as a white solid with a purity >99% as determined by ¹H NMR.

Where a range of numerical values is recited herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In addition, unless explicitly stated otherwise or indicated to the contrary by the context of usage, amounts, sizes, formulations, parameters, and other quantities and characteristics recited herein, particularly when modified by the term “about”, may but need not be exact, and may be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value.

Further in this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

What is claimed is:

1. A process for the preparation of a halogenated aromatic diacid, as described by the structure of Formula (I) 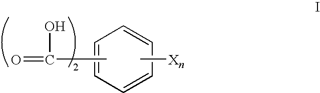
wherein X is Cl, Br or I, and n=1 or 2, comprising the steps of
(a) providing a solution of a catalyst system in a solvent, the catalyst system comprising a cobalt compound, a manganese compound, a zirconium compound, and a bromine compound;
(b) contacting the solution with a halogenated dimethylbenzene, as described by the structure of Formula (III) 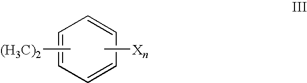
to form a reaction mixture;
(c) stirring the reaction mixture while injecting an oxygen-containing gas therein;
(d) heating the reaction mixture at a first temperature to oxidize one of the two methyl groups of the halogenated dimethylbenzene to produce a compound as described by the structure of Formula (IV); and 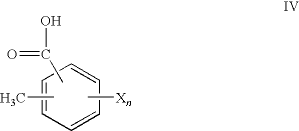
(e) heating the reaction mixture at a second temperature that is higher than the first temperature to oxidize the methyl group in the compound of Formula (IV) to produce a halogenated aromatic diacid.

2. The process of claim 1 wherein the cobalt compound comprises one or more members of the group consisting of cobalt acetate, cobalt naphthenate, cobalt 2-ethylhexanoate, and cobalt bromide.

3. The process of claim 1 wherein the cobalt compound is used in an amount of from about 0.1 to about 5 mol % based on moles of halogenated dimethylbenzene.

4. The process of claim 1 wherein the manganese compound comprises one or more members of the group consisting of manganese acetate, manganese naphthenate, manganese 2-ethylhexanoate, and manganese bromide.

5. The process of claim 1 wherein the manganese compound is used in an amount of from about 0.1 to about 5 mol % based on moles of halogenated dimethylbenzene.

6. The process of claim 1 wherein the zirconium compound comprises one or more members of the group consisting of zirconium acetate, zirconium naphthenate, zirconium 2-ethylhexanoate, and zirconium bromide.

7. The process of claim 1 wherein the zirconium compound is used in an amount of from about 0.01 to about 0.5 mol % based on moles of halogenated dimethylbenzene.

8. The process of claim 1 wherein the bromine compound comprises one or more members of the group consisting of sodium bromide, potassium bromide, hydrogen bromide, bromine, cobalt bromide, manganese bromide, zirconium bromide, and tetrabromoethane.

9. The process of claim 1 wherein the bromine compound is used in an amount of from about 0.2 to about 8 mol % based on moles of halogenated dimethylbenzene.

10. The process of claim 1 wherein the mole ratio of cobalt compound:manganese compound:zirconium compound:bromine compound is about 1:1-1.5:0.05-0.2:1-3.

11. The process of claim 1 wherein the cobalt compound comprises cobalt acetate, the manganese compound comprises manganese acetate, the zirconium compound comprises zirconium acetate, and the bromine compound comprises sodium bromide or potassium bromide.

12. The process of claim 11 wherein the mole ratio of cobalt acetate:manganese acetate:zirconium acetate:sodium or potassium bromide is about 1:1:0.1:2.

13. The process of claim 1 wherein the catalyst system consists essentially of a cobalt compound, a manganese compound, a zirconium compound, and a bromine compound.

14. The process of claim 1 wherein the solvent comprises a monocarboxylic acid.

15. The process of claim 1 wherein the first reaction temperature is from about 120° C. to about 150° C.

16. The process of claim 1 wherein the second temperature is from about 150° C. to about 180° C.

17. The process of claim 1 wherein the second reaction temperature is about 20° C. to about 30° C. higher than the first reaction temperature.

18. The process of claim 1 wherein the halogenated aromatic diacid is selected from the group consisting of 2,5-dibromoterephthalic acid, 2,5-dichloroterephthalic acid, 2-bromoterephthalic acid, 2-chloroterephthalic acid, 2,4-dibromoisophthalic acid, 2,4-dichloroisophthalic acid, 2-bromoisophthalic acid, 2-chloroisophthalic acid, 4-bromoisophthalic acid, 4-chloroisophthalic acid, 5-bromoisophthalic acid, and 5-chloroisophthalic acid.

19. A process according to claim 1 further comprising a step of subjecting a halogenated aromatic diacid to a reaction to prepare therefrom a compound, monomer, oligomer or polymer.

20. A process according to claim 19 wherein a polymer prepared comprises a pyridobisimidazole-2,6-diyl(2,5-dihydroxy-p-phenylene)polymer.
